(19) United States
(12) Patent Application Publication
(10) Pub. No.: US 2005/0203405 A1
(43) Pub. Date: Sep. 15, 2005

(54) Ultrasonic imaging method and apparatus

(75) Inventor: Kazuhiro Tsujita, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/077,123

(22) Filed: Mar. 11, 2005

(30) Foreign Application Priority Data
Mar. 15, 2004 (JP) 2004-072095

(51) Int. Cl. A61B 8/02; A61B 8/06; A61B 8/12

(52) U.S. Cl. 600/455

(57) ABSTRACT

Ultrasonic imaging method and apparatus capable of expressing differences of tissues more clearly by focusing attention on the relationship between characteristics of an object and frequency characteristics of received ultrasonic waves. The apparatus includes an ultrasonic transmitting and receiving unit for transmitting and receiving ultrasonic waves to obtain a detection signal; a frequency component calculating unit for calculating plural frequency components from data based on the detection signal; a tissue discrimination computing unit for obtaining characteristics, which change depending on tissues of the object, based on the calculated plural frequency components; a selector unit for selecting at least one of plural frequency bands corresponding to at least one tissue displayed within one screen in accordance with the obtained characteristics; and an image data generating unit for generating image data representing an ultrasonic image based on intensity of the detection signal in the selected frequency band.

### BACKGROUND OF THE INVENTION

1. Field of the Invention

The present invention relates to ultrasonic imaging method and apparatus for performing imaging of organs, bones, etc. within a living body by transmitting and receiving ultrasonic waves so as to generate ultrasonic images to be used for diagnosis.

2. Description of a Related Art

In an ultrasonic imaging apparatus to be used for medical application, normally, an ultrasonic probe including plural ultrasonic transducers having functions of transmitting and receiving ultrasonic waves is used. Using such an ultrasonic probe, an object to be inspected is scanned by an ultrasonic beam formed by synthesizing plural ultrasonic waves, and the ultrasonic echoes reflected from the object are received, and thereby, image information on the object is obtained based on the intensity of the ultrasonic echoes. Furthermore, two-dimensional or three-dimensional images on the object are reproduced based on the image information.

In the case where an ultrasonic image of an object is generated by transmitting an ultrasonic beam from an ultrasonic probe to a human body and receiving ultrasonic echoes, the deeper the depth of the object reflecting the ultrasonic beam, the higher frequency components of the ultrasonic echoes attenuate. Accordingly, the deeper the depth of the object reflecting the ultrasonic beam, the lower frequency components the ultrasonic image is generated based on. On the other hand, the frequency characteristics of the received ultrasonic waves largely depend not only on the depth of the object, but also on the characteristics of the object. However, the ultrasonic image is not generally generated in consideration of the relationship between the characteristics of the object and the frequency characteristics of the received ultrasonic waves.

By the way, it has been proposed to generate an ultrasonic image based on intensity differences between plural frequency components included in the ultrasonic echoes. However, even in the case, the frequency band to be used is limited within a range usable in one ultrasonic probe or not particularly specified.

As a related technology, Japanese Translation Publication of PCT Patent Application JP-A-9-505745 (International Publication WO95/10229) discloses an improved method and apparatus for non-invasive and quantitative evaluation of bone tissue in vivo. According to the document, a bone is iteratively subjected to an ultrasonic acoustic excitation signal pulse of finite duration supplied from one of two transducers on opposite sides of the bone and involving a composite sine-wave signal consisting of plural discrete frequencies that are spaced in the ultrasonic range to approximately 2 MHz, and the excitation signal is repeated substantially in the range up to 1000 Hz. Further, the given number of successive signals most recently received by the other transducer are sequentially averaged to obtain a Fourier transform of an averaged per-pulse signal. On the other hand, by a separate operation not involving the bone, the same transducers perform the transmission and reception of the same excitation signal via a medium of known acoustic properties and path length so as to generate a reference signal to be processed in order to establish the Fourier transform of the reference signal. The two Fourier transformed signals are comparatively evaluated to produce a bone-transfer function (bone-transmitted function). However, to compare the two Fourier transformed signals, they must be timed and the apparatus becomes complicated. In addition, since the two Fourier transformed signals are compared based on the signals measured in experimental systems with completely different settings, not only bone-specific information but also factors due to setting error and so on influence the signals, and thereby, measurement reliability is low.

Japanese Patent Application Publication JP-A-7-51270 discloses, with respect to an ultrasonic diagnostic apparatus, providing a method of forming an image reflecting a shape of a frequency spectrum of an ultrasonic reflection signal from a living body and means for extracting and drawing information accompanying propagation and attenuation in vivo. This ultrasonic diagnostic apparatus displays images by band-splitting a reception signal by using a group of band filters and performing weighting addition or hue addition of intensity signals independently detected by detectors via logarithmic compression circuits. However, this document does not disclose based on what the weighting addition is performed on the intensity signals.

Japanese Patent Application Publication JP-A-10-146338 discloses an ultrasonic diagnostic apparatus capable of providing information on attenuation together with tissue structure information according to B-mode. This ultrasonic diagnostic apparatus scans a section of an object to be inspected with ultrasonic waves and generating a B-mode image based on the obtained reflection signal, wherein the B-mode image is displayed with light and shade and frequency information of the reflection signal is displayed with color by being superimposed on the B-mode image.

Japanese Translation Publication of PCT Patent Application JP-A-2002-506666 (International Publication WO99/47046) discloses a method and apparatus for constructing and/or using of multi-dimensional fields that can be used for high-resolution detection and characterization of distinct features within three-dimensional objects. Specifically, there is provided construction of such multi-dimensional field renderings for high-resolution detection and identification of medical pathologies in human and animal bodies, especially, cancer in organs and tissues. Further, there is provided detection and characterization of other medical pathologies including pathologies of musculoskeletal systems, digestive systems and gastrointestinal tract in addition to atherosclerosis, sclerosis, atherosclerotic heat disease, myocardial infarction, trauma to arterial or veinal walls.

### SUMMARY OF THE INVENTION

In view of the above points, an object of the present invention is to provide ultrasonic imaging method and apparatus capable of expressing differences of tissues more clearly by focusing attention on the relationship between characteristics of an object and frequency characteristics of received ultrasonic waves.

In order to achieve the above-described object, an ultrasonic imaging method according to the present invention includes the steps of: (a) transmitting ultrasonic waves to an object to be inspected and receiving ultrasonic waves reflected from the object or transmitted through the object to obtain a detection signal; (b) calculating a plurality of frequency components from data based on the detection signal obtained at step (a); (c) obtaining characteristics, which change depending on tissues of the object, based on the plurality of frequency components calculated at step (b); (d) selecting at least one of a plurality of frequency bands corresponding to at least one tissue displayed within one screen in accordance with the characteristics obtained at step (c); and (e) generating image data representing an ultrasonic image of the object based on intensity of the detection signal in the at least one frequency band selected at step (d).

Further, an ultrasonic imaging apparatus according to the present invention includes: ultrasonic transmitting and receiving means for transmitting ultrasonic waves to an object to be inspected and receiving ultrasonic waves reflected from the object or transmitted through the object to obtain a detection signal; frequency component calculating means for calculating a plurality of frequency components from data based on the detection signal obtained by the ultrasonic transmitting and receiving means; tissue discriminating means for obtaining characteristics, which change depending on tissues of the object, based on the plurality of frequency components calculated by the frequency component calculating means; selecting means for selecting at least one of a plurality of frequency bands corresponding to at least one tissue displayed within one screen in accordance with the characteristics obtained by the tissue discriminating means; and image data generating means for generating image data representing an ultrasonic image of the object based on intensity of the detection signal in the at least one frequency band selected by the selecting means.

According to the present invention, by selecting at least one of the plurality of frequency bands of the detection signal corresponding to at least one tissue displayed within one screen in accordance with the characteristics which change depending on tissues of the object, tissue differences can be expressed more clearly.

### BRIEF DESCRIPTION OF THE DRAWINGS

FIG. 1 is a block diagram showing the constitution of an ultrasonic imaging apparatus according to the first embodiment of the present invention;

FIG. 2 shows differences between frequency characteristics in ultrasonic waves depending on plural tissues within a spine;

FIG. 3 is a block diagram showing a modified example of the ultrasonic imaging apparatus according to the first embodiment of the present invention;

FIG. 4 is a schematic diagram showing an ultrasonic imaging apparatus according to the second embodiment of the present invention;

FIG. 5 shows an arrangement example of transmitting element arrays to be used in an ultrasonic transmitting unit and receiving element arrays to be used in an ultrasonic receiving unit of the ultrasonic imaging apparatus as shown in FIG. 4; and

FIG. 6 is a block diagram showing the detailed constitution of a signal processing unit as shown in FIG. 4.

### DESCRIPTION OF THE PREFERRED EMBODIMENTS

Hereinafter, embodiments of the present invention will be described in detail by referring to the drawings. The same reference numbers are assigned to the same component elements and the description thereof will be omitted.

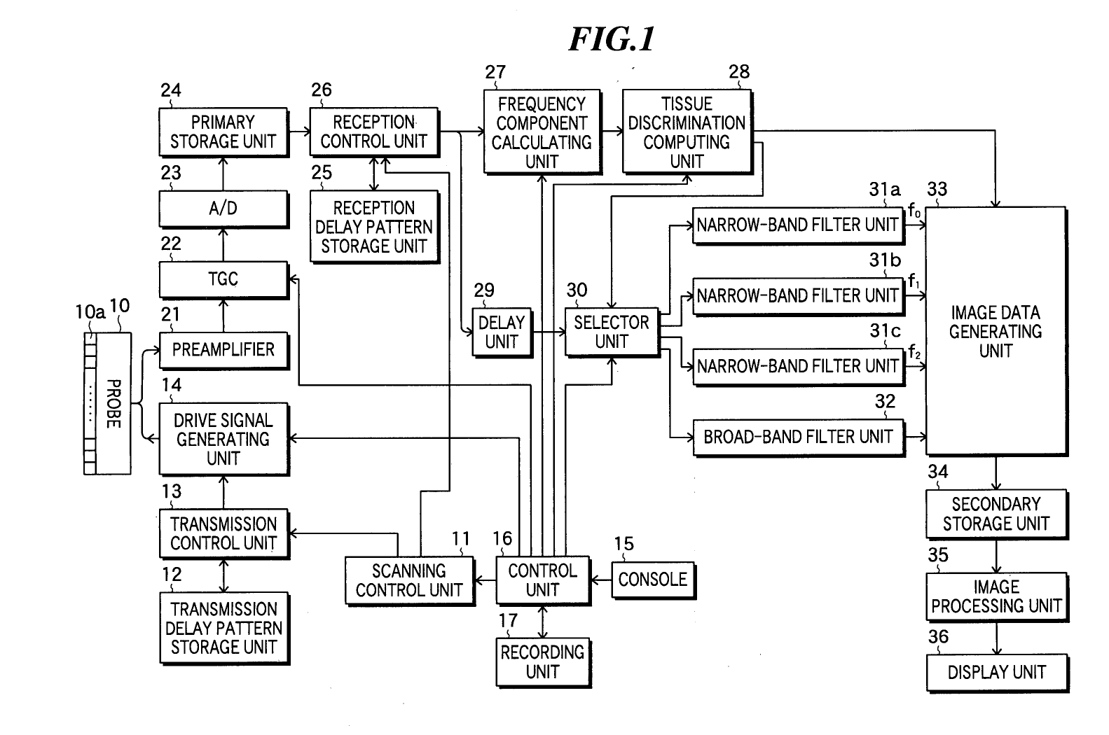

FIG. 1 is a block diagram showing the constitution of an ultrasonic imaging apparatus according to the first embodiment of the present invention. The ultrasonic imaging apparatus according to the embodiment includes an ultrasonic probe 10, a scanning control unit 11, a transmission delay pattern storage unit 12, a transmission control unit 13, and a drive signal generating unit 14.

The ultrasonic probe 10 includes plural ultrasonic transducers 10a arranged in a one-dimensional or two-dimensional manner to form a transducer array, and the ultrasonic probe 10 is used by being abutted on an object to be inspected. These ultrasonic transducers 10a transmit ultrasonic beams based on applied drive signals, and receive propagating ultrasonic echoes to output detection signals.

Each ultrasonic transducer is constituted by a vibrator in which electrodes are formed on both ends of a material having a piezoelectric property (piezoelectric material) such as a piezoelectric ceramic represented by PZT (Pb (lead) zirconate titanate), a polymeric piezoelectric element represented by PVDF (polyvinylidene difluoride), or the like. When a voltage is applied to the electrodes of the ultrasonic transducer by transmitting pulse electric signals or continuous wave electric signals, the piezoelectric element expands and contracts. By the expansion and contraction, pulse ultrasonic waves or continuous wave ultrasonic waves are generated from the respective ultrasonic transducers, and an ultrasonic beam is formed by synthesizing these ultrasonic waves. Further, the respective ultrasonic transducers expand and contract by receiving propagating ultrasonic waves to generate electric signals. These electric signals are outputted as detection signals of ultrasonic waves.

Alternatively, as the ultrasonic transducers, elements of different materials may be used for transmission and reception, or plural kinds of elements of different conversion types may be used. For example, the above-described ultrasonic transducers are used as elements for transmitting ultrasonic waves and photo-detection type ultrasonic transducers are used as elements for receiving ultrasonic waves. The photo-detection type ultrasonic transducer is for detecting ultrasonic waves by converting ultrasonic signals into optical signals, and constituted by a Fabry-Perot resonator or fiber Bragg grating, for example.

The scanning control unit 11 sets the transmission direction of ultrasonic beams and the reception direction of ultrasonic echoes sequentially. The transmission delay pattern storage unit 12 has stored plural transmission delay patterns to be used when ultrasonic beams are formed. The transmission control unit 13 selects a suitable pattern from the plural delay patterns that have been stored in the transmission delay pattern storage unit 12 in accordance with the transmission direction set in the scanning control unit 11, and sets delay times of drive signals to be respectively provided to the plural ultrasonic transducers 10a based on the pattern.

The drive signal generating unit 14 includes a signal generator circuit for generating predetermined signals and plural drive circuits for respectively outputting plural drive signals to be supplied to the plural ultrasonic transducers 10a by providing delays to the signals generated by the signal generator circuit. The signal generator circuit generates signals having plural frequency components such as frequency multiple signals, generates signals having frequencies that change over time such as chirp signals or sweep signals, or burst signals having frequencies sequentially changed at each time of transmission. The drive circuits delay the signals generated by the signal generator circuit based on the delay times set in the transmission control unit 13. Ultrasonic waves are transmitted from the plural ultrasonic transducers 10a according to the drive signals outputted from the drive signal generating unit 14.

Further, the ultrasonic imaging apparatus according to the embodiment includes a console 15, a control unit 16 having a CPU, and a recording unit 17 such as a hard disk. The control unit 16 controls the scanning control unit 11, the drive signal generating unit 14, a frequency component calculating unit 27, a tissue discrimination component unit 28, a selector unit 29, and so on based on the operation by an operator using the console 15. In the recording unit 17, there are recorded programs for activating the CPU that constitutes the control unit 16 to execute various kinds of operation, and data on conversion efficiency and frequency characteristics in transmission and reception of the ultrasonic transducers.

Furthermore, the ultrasonic imaging apparatus according to the embodiment includes a preamplifier 21, a TGC (time gain compensation) amplifier 22, an A/D (analog/digital) converter 23, a primary storage unit 24, a reception delay pattern storage unit 25, a reception control unit 26, the frequency component calculating unit 27, the tissue discrimination computing unit 28, a delay unit 29, a selector unit 30, narrow-band filter units 31a, 31b and 31c, abroad-band filter unit 32, an image data generating unit 33, a secondary storage unit 34, an image processing unit 35, and a display unit 36.

Ultrasonic beams transmitted from the ultrasonic probe 10 are reflected from an object to be inspected and thus generated ultrasonic echoes are received by the plural ultrasonic transducers 10a, and thereby, plural detection signals are generated. These detection signals are amplified by the preamplifier 21, and subjected to attenuation correction in response to the distance that ultrasonic waves reach within the object by the TGC amplifier 22. Further, variations in conversion efficiency and frequency characteristics between the plural ultrasonic transducers are corrected by using data recorded in the recording unit 17.

The detection signals outputted from the TGC amplifier 22 are converted into digital signals by the A/D converter 23. As a sampling frequency of the A/D converter 23, at least about a tenfold frequency of that of the ultrasonic wave is required, and a 16-fold or more frequency of that of the ultrasonic wave is desirable. Further, as the resolving power of the A/D converter 23, a resolving power of ten or more bits is desirable. The primary storage unit 24 stores the digital detection signals outputted from the A/D converter 23 with respect to each ultrasonic transducer in chronological order.

The reception delay pattern storage unit 25 has stored plural reception delay patterns to be used when reception focusing processing is performed on the plural detection signals outputted from the plural ultrasonic transducers 10a. The reception control unit 26 performs reception focusing processing by selecting a suitable pattern from the plural reception delay patterns that have been stored in the reception delay pattern storage unit 25 based on the reception direction set in the scanning control unit 11, and providing delays to the plural detection signals based on the pattern and adding the signals to each others. By the reception focusing processing, ultrasound beam data is formed which represents an ultrasound beam signal as a detection signal in which the focus of the ultrasonic echo is narrowed. By the way, the reception focusing processing may be performed before the A/D conversion or the correction by the TGC amplifier 22.

The frequency component calculating unit 27 calculates plural frequency components included in the ultrasound beam signal (detection signal) based on the ultrasound beam data outputted from the reception control unit 26. Here, a reference frequency f₀ and plural frequencies f₁, f₂, . . . to be used for obtaining differences from the reference frequency component are set by focusing attention on the relationship between characteristics of the object and frequency characteristics of the received ultrasonic waves.

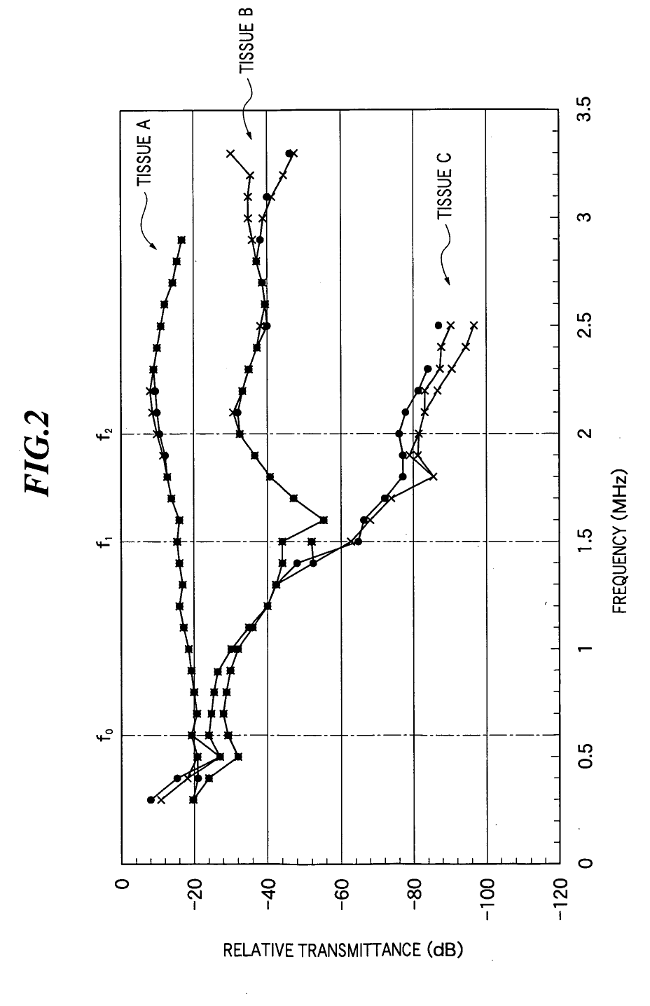

FIG. 2 shows differences between frequency characteristics in ultrasonic waves depending on plural tissues within a spine. As shown in FIG. 2, the frequency characteristics in relative transmittances of ultrasonic waves largely differ depending on tissues. Specifically, tissue A (nucleus pulposus) has a frequency characteristic flat over a broad band. However, tissue B (bone containing soft tissue) has a dip around 1.5 MHz, and tissue C (bone) has a frequency characteristic that continues high-frequency attenuation even at 2 MHz. In view of such differences in frequency characteristics, in the embodiment, the reference frequency f₀ is set to 0.6 MHz in the range from 0.5 MHz to 0.7 MHz in which relative transmittance change depending on the tissue difference is small, the frequency f₁ is set to 1.5 MHz, and the frequency f₂ is set to 2 MHz. Alternatively, the operator may input desired values of these frequencies by using the console 15 as shown in FIG. 1, and the control unit 16 may set these values in the frequency component calculating unit 27 and the tissue discrimination computing unit 28.

Referring to FIG. 1 again, the tissue discrimination computing unit 28 obtains tissue characteristics (characteristics that change depending on tissues) such as frequency characteristics in attenuation factors of ultrasonic waves by computation based on the plural frequency components calculated by the frequency component calculating unit 27. For example, the tissue discrimination computing unit 28 obtains frequency attenuation factors {I (f₁)−I(f₀)} and {I (f₂)−I(f₀)} as intensity differences between the plural frequency components calculated by the frequency component calculating unit 27, or obtains differences in attenuation factors {I(f₁)−I(f₀)}−{I(f₂)−I(f₀)}, {I(f₁)−I(f₀)}−{I(f₂)−I(f₁)}, or the like. In the above expression, the intensity of frequency component is expressed in decibels. In the case where the intensity of frequency component is expressed in voltages, intensity ratios of frequency components are used as frequency attenuation factors and ratios of the frequency attenuation factors are used in place of differences between frequency attenuation factors. The tissue discrimination computing unit 28 outputs a control signal to be used for selecting one of plural frequency bands according to thus obtained tissue characteristic.

Further, the ultrasound beam data outputted from the reception control unit 26 is selectively supplied to at least one of the narrow-band filter units 31a, 31b and 31c and the broad-band filter unit 32 via the delay unit 29 and the selector unit 30. The narrow-band filter units 31a to 31c constitute band-pass filters for allowing plural different narrow band signals to pass, respectively, and, for example, center frequencies of the pass bands are set to f₀, f₁ and f₂, respectively. Further, the broad-band filter unit 32 forms a band-pass filter for allowing broad band signals to pass.

The tissue discrimination computing unit 28 outputs a control signal obtained with respect to a certain line after ultrasound beam data of that line is inputted a predetermined period (e.g., a period for one line) behind. The delay unit 29 delays the ultrasound beam data outputted from the reception control unit 26 by a predetermined period (e.g., a period for one line) in order to absorb the time lag. The selector unit 30 selects at least one of the narrow-band filter units 31a to 31c according to the control signal outputted from the tissue discrimination computing unit 28, and supplies the ultrasound beam data to the selected narrow-band filter unit.

The selected narrow-band filter unit performs narrow-band band-pass filter processing on the supplied ultrasound beam data, and thereby, makes ultrasound beam signals represented by the ultrasound beam data to fall within narrow-bands. On the other hand, when a normal ultrasonic image is displayed, under the control of the control unit 16, the selector unit 30 selects, for example, the broad-band filter unit 32 having a pass band nearly including the transmission wave bands.

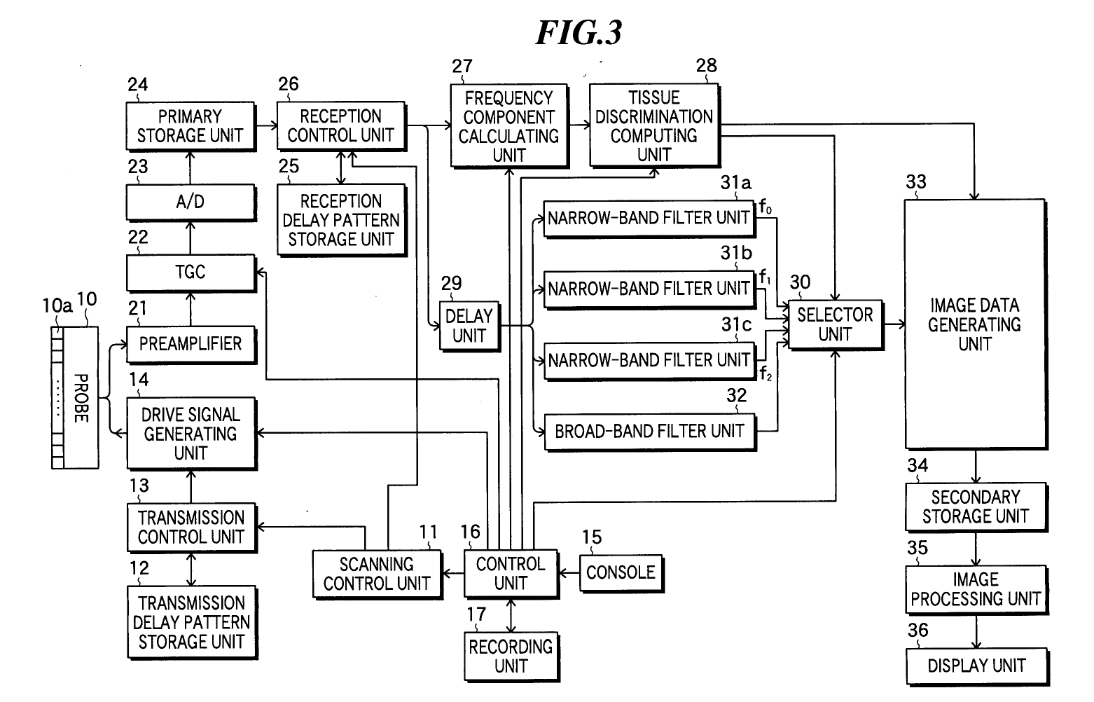

Alternatively, as shown in FIG. 3, the order of the selector unit and filter units may be changed so that the selector unit 30 selects one kind of ultrasound beam data outputted from the narrow-band filter units 31a to 31c and the broad-band filter unit 32 and supplies the selected data to the image data generating unit 33.

The ultrasound beam data that has been subjected to the filter processing is inputted to the image data generating unit 33. The image data generating unit 33 performs various kinds of signal processing such as peak value detection, RMS value detection, envelope detection and data interpolation between ultrasound beams based on the ultrasound beam data that has been subjected to the filter processing so as to generate B-mode image data.

Thus, according to the tissue characteristics obtained in the tissue discrimination computing unit 28, at least one frequency band suitable for at least one tissue displayed within one screen of the ultrasonic image is selected, and image data representing the ultrasonic image of the object is generated based on the intensity of the ultrasound beam signals (detection signals) in the frequency band. For example, a tissue having a larger ultrasonic wave attenuation factor may be displayed by using an ultrasound beam signal having a lower frequency. As described above, by selecting at least one frequency band of the detection signals in correspondence with at least one tissue displayed within one screen, tissue differences can be expressed more clearly than by the conventional one.

Furthermore, the image data generating unit 33 can change color information of the ultrasonic image in correspondence with the plural tissues displayed within one screen based on the tissue characteristics obtained in the tissue discrimination computing unit 28. In this case, the tissue difference can be discriminated by the changes in color. For example, the image data generating unit 33 may add color to the ultrasonic image according to the control signal outputted from the tissue discrimination computing unit 28. Alternatively, the image data generating unit 33 may sequentially or simultaneously perform operation of generating image data of R (red) based on the ultrasound beam data outputted from the narrow-band pass filter unit 31a, generating image data of G (green) based on the ultrasound beam data outputted from the narrow-band pass filter unit 31b, and generating image data of B (blue) based on the ultrasound beam data outputted from the narrow-band pass filter unit 31c, and combine three kinds of images respectively represented by the image data of RGB.

The secondary storage unit 34 stores image data outputted from the image data generating unit 33. The image processing unit 35 performs various kinds of image processing on the image data stored in the secondary storage unit 34. The display unit 36 includes a display device such as a CRT, an LCD, or the like, and displays ultrasonic images based on the image signal that has been subjected to image processing by the image processing unit 35.

As described above, in the embodiment, the plural frequency components are obtained from the ultrasound beam signals, and tissue characteristics such as frequency characteristics of attenuation factors of ultrasonic waves are obtained based on these frequency components, and therefore, detailed tissue discrimination can be performed. As a result, in correspondence with the obtained tissue characteristics, an ultrasonic image can be obtained based on the ultrasound beam signals having different frequencies. Thereby, tissue differences are clarified, and further, an ultrasonic image having different speckle size from a normal ultrasonic image can be obtained.

Here, the speckle refers to a portion in which light points and dark points are scattered due to interference of a series of ultrasonic waves reflected by a large number of microstructures. The speckle is a kind of artifacts, however, it can be interpreted to express the change in quality at the area of a lesion. Therefore, by displaying the image by varying the brightness and speckle size more clearly according to the tissue characteristics than in the conventional one, the respective tissue differences and lesion can be discriminated more easily.

Further, in the embodiment, differences or ratios between the reference frequency components and other frequency components are obtained from the detection signals obtained by receiving ultrasonic echoes, unlike comparison between levels of ultrasonic waves that have passed through plural paths, and therefore, an ultrasonic image corresponding to tissue characteristics can be obtained without being affected by contour factors such as the surface form of the object and incident angle of ultrasonic wave to the surface.

Next, the second embodiment of the present invention will be described. The second embodiment of the present invention is to apply the present invention to an ultrasonic CT (computerized tomography). The ultrasonic CT is an image construction method by setting some mathematical model between an acoustic physical parameter of an object such as sound speed or attenuation factor and observation data of scattered wave caused by acoustic wave application, and obtaining a physical parameter distribution as a solution of the inverse problem thereof by computation of a computer. Images with high resolving power by the ultrasonic CT have been reported.

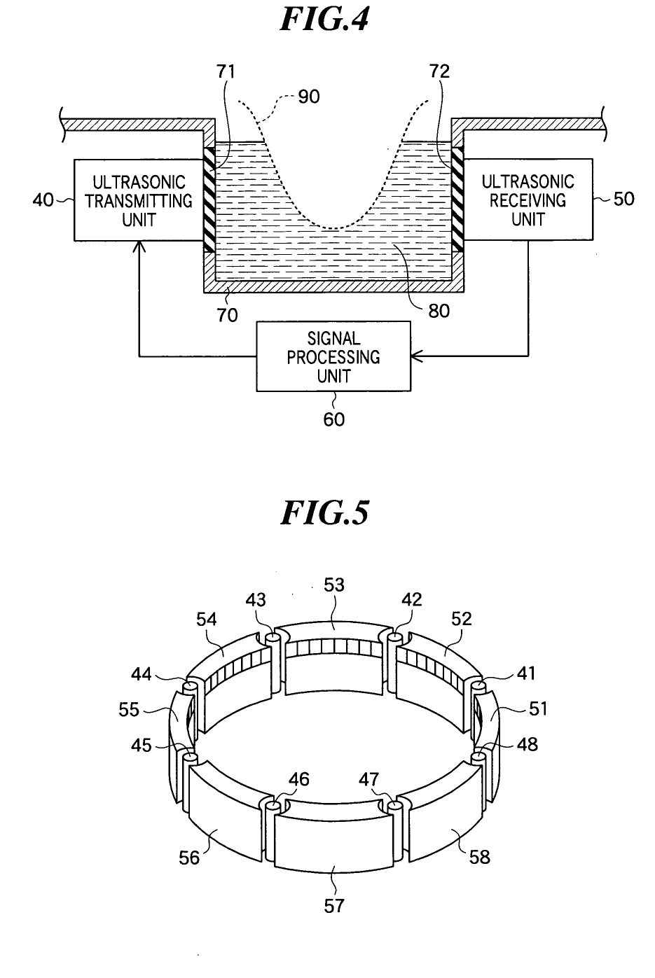

FIG. 4 is a schematic diagram showing an ultrasonic imaging apparatus according to the second embodiment of the present invention. This ultrasonic imaging apparatus is an ultrasonic CT for mammary cancer diagnosis, and includes an ultrasonic transmitting unit 40 for transmitting ultrasonic waves to an object to be inspected (breast of a patient) 90, an ultrasonic receiving unit 50 for receiving ultrasonic waves transmitted through the object 90, a signal processing unit 60 connected to the ultrasonic transmitting unit 40 and ultrasonic receiving unit 50, and a tank 70 in which water or other acoustic medium 80 is put. In the tank 70, acoustic windows 71 and 72 are provided in order to obtain the match of acoustic impedance between the ultrasonic transmitting unit 40 and ultrasonic receiving unit 50 and the acoustic medium 80.

FIG. 5 shows an arrangement example of transmitting element arrays to be used for the ultrasonic transmitting unit and receiving element arrays to be used for the ultrasonic receiving unit of the ultrasonic imaging apparatus as shown in FIG. 4. As shown in FIG. 5, plural transmitting element arrays 41 to 48 and plural receiving element arrays 51 to 58 are arranged along the circumference of the tank 70. In this example, the transmitting element arrays 41 to 48 are formed by plural transmitting elements arranged along the vertical direction, and the receiving element arrays 51 to 58 are formed by plural receiving elements arranged along the circumferential direction.

Ultrasonic waves transmitted from the plural transmitting elements included in the respective transmitting element arrays 41 to 48 form ultrasonic beams. The object is scanned by the ultrasonic beams sequentially transmitted from the transmitting element arrays 41 to 48. The ultrasonic beams transmitted through the object are scattered and received by the plural receiving elements included in the respective receiving element arrays 51 to 58, and thereby, plural detection signals are outputted. Here, the sound speed of the ultrasonic wave can be obtained from the time elapsed between transmission of the ultrasonic wave by the transmitting element and reception of the ultrasonic wave by the receiving element, and from the positional relationship between the transmitting element that transmits the ultrasonic wave and the receiving element that receives the ultrasonic wave.

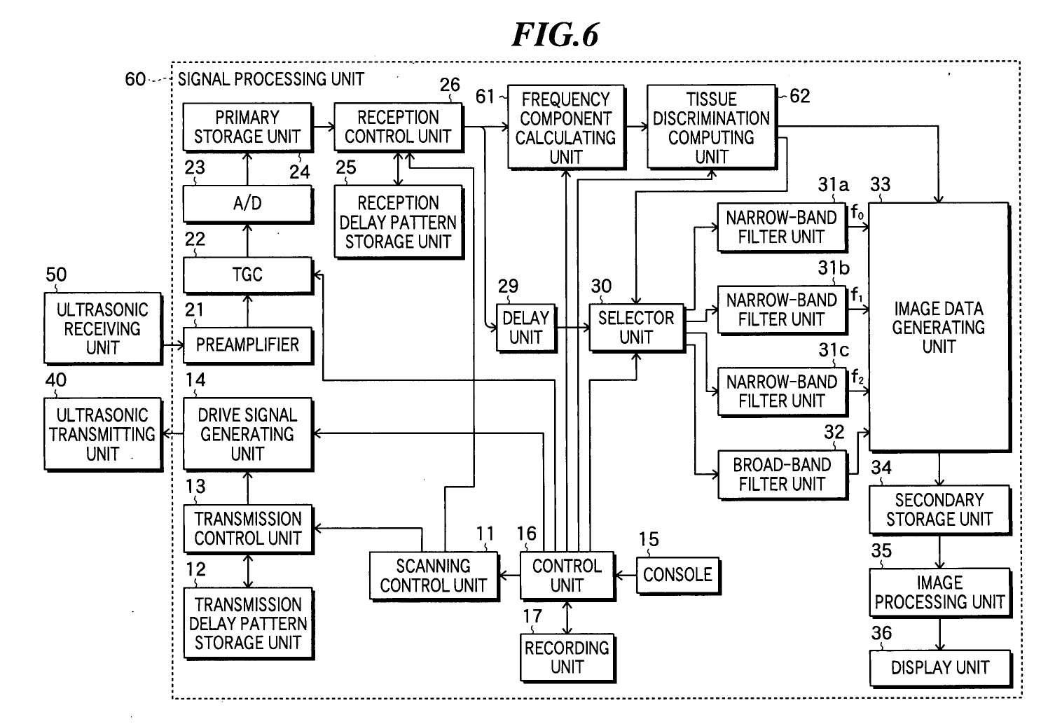

FIG. 6 is a block diagram showing the detailed constitution of the signal processing unit as shown in FIG. 4. The constitution of the signal processing unit 60 is in partially common with that shown in FIG. 1.

A frequency component calculating unit 61 calculates plural frequency components included in the ultrasound beam signal (detection signal) based on the ultrasound beam data outputted from the reception control unit 26. Here, a reference frequency f₀ and plural frequencies f₁, f₂, . . . to be used for obtaining differences from the reference frequency component are set by focusing attention on the relationship between characteristics of the object and frequency characteristics of received ultrasonic waves.

A tissue discrimination computing unit 62 obtains tissue characteristics (characteristics that change depending on tissues) such as transmittances of ultrasonic waves and frequency characteristics in a sound speed by computation based on the plural frequency components calculated by the frequency component calculating unit 61. For example, the tissue discrimination computing unit 62 obtains frequency transmittances {I (f₁)−I(f₀)} and {I (f₂)−I(f₀)} as intensity differences between the plural frequency components calculated by the frequency component calculating unit 61 and differences in frequency transmittances {I(f₁)−I(f₀)}-{I (f₂)−I(f₀)}, {I (f₁)−I(f₀)}-{I (f₂)−I(f₁)}, or the like. In the above expression, the intensity of frequency component is expressed in decibels. In the case where the intensity of frequency component is expressed in voltages, intensity ratios of frequency components are used as frequency transmittances and ratios of the frequency transmittances are used in place of differences between frequency transmittances.

Alternatively, the tissue discrimination computing unit 62 obtains sound speed ratios of ultrasonic waves v(f₁)/v(f₀) and v (f₂)/v (f₀) in the plural frequency components calculated by the frequency component calculating unit 61, the ratio of sound speed ratios {v(f₁)/v(f₀)}/{v(f₂)/v(f₀)} or {v(f₁)/v(f₀)}/{v(f₂)/v(f₁)}, or the like. The tissue discrimination computing unit 62 outputs a control signal for selecting at least one of plural frequency bands to the selector unit 30 according to thus obtained tissue characteristic.

By the selector unit 30, at least one of the narrow-band filter units 31a to 31c having frequency bands suitable for at least one tissue displayed within one screen of the ultrasonic image are selected, and image data representing the ultrasonic image of the object is generated based on the intensity of the ultrasound beam signals (detection signals) in the frequency band. For example, a tissue having a larger ultrasonic wave attenuation factor may be displayed by using an ultrasound beam signal having a lower frequency. As described above, by selecting the frequency bands of the detection signals in response to the plural tissues displayed within one screen, tissue differences can be expressed more clearly than by the conventional one.

By the way, similarly in the second embodiment, as shown in FIG. 3, the order of the selector unit and filter units may be changed so that the selector unit 30 selects one kind of ultrasound beam data outputted from the narrow-band filter units 31a to 31c and the broad-band filter unit 32 and supplies the data to the image data generating unit 33.

1. An ultrasonic imaging method comprising the steps of:
(a) transmitting ultrasonic waves to an object to be inspected and receiving ultrasonic waves reflected from the objector transmitted through the object to obtain a detection signal;
(b) calculating a plurality of frequency components from data based on the detection signal obtained at step (a);
(c) obtaining characteristics, which change depending on tissues of the object, based on the plurality of frequency components calculated at step (b);
(d) selecting at least one of a plurality of frequency bands corresponding to at least one tissue displayed within one screen in accordance with the characteristics obtained at step (c); and
(e) generating image data representing an ultrasonic image of the object based on intensity of the detection signal in the at least one frequency band selected at step (d).

2. The ultrasonic imaging method according to claim 1, wherein step (a) includes simultaneously or sequentially transmitting the plurality of frequency components of the ultrasonic waves.

3. The ultrasonic imaging method according to claim 1, wherein step (c) includes obtaining one of attenuation factors and transmittances of the ultrasonic waves or frequency characteristics in a sound speed based on the plurality of frequency components calculated at step (b).

4. The ultrasonic imaging method according to claim 1, wherein step (c) includes obtaining one of attenuation factors and transmittances of the ultrasonic waves or frequency characteristics in a sound speed based on a reference frequency component and other frequency components calculated at step (b).

5. The ultrasonic imaging method according to claim 4, wherein step (b) includes calculating the plurality of frequency components including the reference frequency component within a range from 0.5 MHz to 0.7 MHz.

6. The ultrasonic imaging method according to claim 1, wherein step (e) includes changing color information of the ultrasonic image in correspondence with a plurality of tissues displayed within one screen based on the characteristics obtained at step (c).

7. An ultrasonic imaging apparatus comprising:
ultrasonic transmitting and receiving means for transmitting ultrasonic waves to an object to be inspected and receiving ultrasonic waves reflected from the object or transmitted through the object to obtain a detection signal;
frequency component calculating means for calculating a plurality of frequency components from data based on the detection signal obtained by said ultrasonic transmitting and receiving means;
tissue discriminating means for obtaining characteristics, which change depending on tissues of the object, based on the plurality of frequency components calculated by said frequency component calculating means;
selecting means for selecting at least one of a plurality of frequency bands corresponding to at least one tissue displayed within one screen in accordance with the characteristics obtained by said tissue discriminating means; and
image data generating means for generating image data representing an ultrasonic image of the object based on intensity of the detection signal in the at least one frequency band selected by said selecting means.

8. The ultrasonic imaging apparatus according to claim 7, wherein said ultrasonic transmitting and receiving means simultaneously or sequentially transmits the plurality of frequency components of the ultrasonic waves.

9. The ultrasonic imaging apparatus according to claim 7, wherein said tissue discriminating means obtains one of attenuation factors and transmittances of the ultrasonic waves or frequency characteristics in a sound speed based on the plurality of frequency components calculated by said frequency component calculating means.

10. The ultrasonic imaging apparatus according to claim 7, wherein said tissue discriminating means obtains one of attenuation factors and transmittances of the ultrasonic waves or frequency characteristics in a sound speed based on a reference frequency component and other frequency components calculated by said frequency component calculating means.

11. The ultrasonic imaging apparatus according to claim 10, wherein said frequency component calculating means calculates the plurality of frequency components including the reference frequency component within a range from 0.5 MHz to 0.7 MHz.

12. The ultrasonic imaging apparatus according to claim 7, wherein said image data generating means changes color information of the ultrasonic image in correspondence with a plurality of tissues displayed within one screen based on the characteristics obtained by said tissue discriminating means.
